(19) United States
(12) Patent Application Publication
(10) Pub. No.: US 2008/0193378 A1
(43) Pub. Date: Aug. 14, 2008

(54) Radio Metal Complexes Based on Bispidine and the Derivatives Thereof as Chelating Agents, and Use Thereof for Nuclear Medical Diagnosis and Therapy

(76) Inventors: Peter Comba, Wiesenbach (DE); Stefanie Juran, Kostebrau (DE); Marion Kerscher, Edingen (DE); Hans-Jurgen Pietzsch, Heidenau (DE); Hartmut Spies, Dresden (DE); Holger Stephan, Dresden (DE)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 11/719,000

(22) PCT Filed: Dec. 7, 2005
(86) PCT No.: PCT/EP2005/013171

§ 371 Date: Nov. 29, 2007

(30) Foreign Application Priority Data
Dec. 24, 2004 (DE) 10 2004 062 568.9

(51) Int. Cl. A61K 51/04

(52) U.S. Cl. 424/1.65

(57) ABSTRACT

The invention relates to specific radioactive metal complexes based on bispidine and the derivatives thereof as chelating agents, the use thereof for nuclear medical diagnosis and therapy, and a method for producing such radioactive metal complexes.

The invention relates to specific radioactive metal complexes based on bispidine and the derivatives thereof as chelating agents, and use thereof for nuclear medical diagnosis and therapy, and to a method for preparing such radioactive metal complexes.

Nuclear medicine, as an important branch of modern medicine, makes use of open radioactive substances, called radiopharmaceuticals, for diagnostic and therapeutic purposes. The number of radionuclides having radiation properties of diagnostic and therapeutic use which, by their chemical nature, are metals predominates, and therefore studies on the development of metalloradiopharmaceuticals play an important part in modern radiopharmaceutical research (C. J. Anderson, M. J. Welch: “Radiometal-labeled agents (non-technetium) for diagnostic imaging”. Chem. Rev. 99 (1999) 2219-2234; W. A. Volkert, T. J. Hoffmann: “Therapeutic radiopharmaceuticals”. Chem. Rev. 99 (1999) 2269-2292).

A substantial limiting factor in the development of metalloradiopharmaceuticals in general and in particular also for copper radiopharmaceuticals is the lack of suitable complexing agents with which the radioactive metal can be stably coupled to biomolecules. In particular, the stability of many metal chelates to hydrolysis, which proceeds with liberation of the radioactive metal, is inadequate.

Copper isotopes are of interest both for diagnostic and therapeutic use in endoradiotherapy. This relates to the positron emitters ⁶⁰Cu, ⁶¹Cu, ⁶²Cu and ⁶⁴Cu for positron emission tomography (PET) and ⁶⁴Cu and the β⁻-emitting ⁶⁷Cu (Eₘₐₓ=0.576 MeV) as attractive radioisotopes for radiotherapy and radioimmunotherapy (W. A. Volkert, T. J. Hoffmann loc.cit.; J. S. Lewis, M. J. Welch: “Copper Chemistry related to Radiopharmaceutical Production”. In: M. Nicolini, U. Mazzi (Ed.) Proc. of 6th. Intern. Symp. on Technetium in Chemistry and Nuclear Medicine, (Bressanone, September 2002) p. 23-33).

The nuclide properties of ⁶⁴Cu allow it to be used for PET imaging and radiotherapy. ⁶⁴Cu-labeled radio-pharmaceuticals therefore allow radiotherapy with simultaneous monitoring of the distribution and biokinetics by PET imaging. The nuclide can be obtained in good yields and high specific activity in small cyclotrons and is therefore available at low cost for routine preparation of radiopharmaceuticals for the treatment for example of neoplastic diseases.

Despite this, only a few radiopharmaceuticals based on radioactive copper isotopes are yet available. Examples are in the first place ⁶⁰Cu-ATSM as hypoxia marker, and ⁶⁴Cu-ATSM and ⁶⁴Cu-PTSM, which are suggested as potential agents for tumor therapy. Further classes of substances are copper-labeled peptides and antibodies in which the radioactive copper is linked to the biomolecule via a bifunctional chelator.

As already stated in relation to metalloradiopharmaceuticals, the development of copper radiopharmaceuticals also requires suitable complexing agents with which hydrolysis-stable and radiochemically stable linkage of the radioactive metal is possible. The currently inadequate availability of such complexing agents is a serious limiting factor in the development of copper radiopharmaceuticals. Studies on the development of copper-labeled antibodies have revealed that cyclic tetraaza compounds are the most frequently used complexing agents (P. J. Blower, J. S. Lewis, J. Zweit: “Copper Radionuclides and Radiopharmaceuticals in Nuclear Medicine”, Nucl. Med. Biol. 23 (1996) 957-980; D. Parker: “Imaging and Targeting” in Comprehensive Supramolecular Chemistry, vol. 10 “Supra-molecular Technology”, J. L. Atwood, J. E. D. Davis, D. P. Mac Nicol, F. Vögtle, J.-M. Lehn (Ed.), Pergamon 1996, 487-536). However, these compounds have also proved to be unsuitable in particular for therapeutic use because their effects are too non-specific.

The basic requirement to be met by metal complexes which can be employed with prospects of success as radiopharmaceuticals are very high complex stabilities and metabolic stability in relation to the chelate. Since the choice of the complexing agent drastically influences the biokinetics, distribution and metabolism of the radioactive compound, the complexing agent should additionally be amenable to structural variation in order to be able to optimize biodistribution patterns of the metal-biomolecule conjugates.

The present invention is thus based on the object of providing radioactive metal complexes in which the radioactive metals are bound with high stability, and which are metabolically stable in the body after administration.

This subject is achieved by the embodiments characterized in the claims.

In particular, radioactive metal complexes based on bispidine and the derivatives thereof (bispidine=3,7-diazabicyclo[3.3.1]nonane) as chelating agents having the following general formula (I)

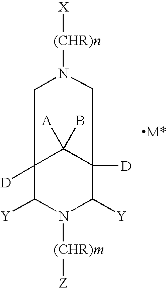

in which
A and B are independently of one another H, —OR¹, —SR¹, —NHR¹, —OC(O)R¹, —NHC(O)R¹ or a straight-chain or branched-chain (C₁₋₈) alkyl radical, or A and B together are ═O or ═S, where R¹ is hydrogen, a straight-chain or branched-chain (C₁₋₈) alkyl radical, a substituted or unsubstituted (C₆₋₁₀) aryl radical, in particular phenyl, D is in each case a carboxyl group or a derivative derived therefrom and selected from esters, amides and peptides,
X, Y and Z are independently of one another an unsubstituted or substituted heterocycle selected from pyridine, pyrimidine, pyrazine, thiophene, furan, pyrazole, imidazole, thiazole, quinoline, quinazoline and quinoxaline, where the substituents may have been selected from straight-chain or branched-chain (C₁₋₈) alkyl radicals, halogen, straight-chain or branched-chain (C₁₋₈) alkoxy radicals, sulfonate, carboxylate, nitro, cyano, hydroxy, benzyl or phenyl,
the radical R in the respective alkylene bridge is independently of one another hydrogen, hydroxy, a straight-chain or branched-chain (C₁₋₈) alkyl radical, a straight-chain or branched-chain (C₁₋₈) alkoxy radical, —OC(O)R¹ or —NHC(O)R¹, where the radical R¹ is as defined above,
M* is a radioactive metal, and
m and n is in each case an integer between 1 and 5, are provided.

A further aspect of the present invention relates to the use of the radioactive metal complexes of the invention for diagnosis and therapy, and a method for preparing such radioactive metal complexes.

It is possible to employ as the radioactive metal M* in the context of the present invention in particular a nuclide of copper, of rare earths, Tc, In, Ga, Y or Re. The radioactive metal M* can preferably be selected from ⁶⁴Cu or ¹⁸⁸Re.

In a preferred embodiment of the present invention, metal complexes where, in formula (I), A and B are independently of one another H, —OR¹, —OC(O)R¹ or —NHC(O)R¹, or A and B are together ═O,

D is in each case a carboxyl group or an ester derivative derived therefrom,
X, Y and Z are in each case an unsubstituted or substituted pyridine, and
the radical R in the respective alkylene bridge is independently of one another hydrogen, hydroxy, a straight-chain or branched-chain (C₁₋₈) alkyl radical, a straight-chain or branched-chain (C₁₋₈) alkoxy radical, —OC(O)R¹ or —NHC(O)R¹, where the radical R¹ is as defined above,
are provided.

In a further preferred embodiment of the present invention, metal complexes where, in formula (I), A and B are respectively H and OH,

D is in each case a —COOH group or a —COOMe group,
X, Y and Z are in each case 2-pyridinyl, and
the radical R in the respective alkylene bridge is in each case hydrogen,
and m and n are each 1,
are provided.

Bispidines having two tertiary amine and four pyridine donors on a very rigid structure derived from diazaadamantane are in particular regarded as efficient ligands in particular for, for example, copper, whose resulting complexes are exceptionally stable.

An important feature of the compounds of the invention is that they exhibit a chemical and radiolytic stability which is improved by comparison with complexes available in the state of the art. In addition, the basic bispidine structure provides diverse possibilities for introducing biomolecules.

The present invention is further illustrated by the following, non-limiting examples. In these, the two specific chelating agents derived from bispidin-9-ol and bispidin-9-ol dicarboxylic acid are used as representatives.

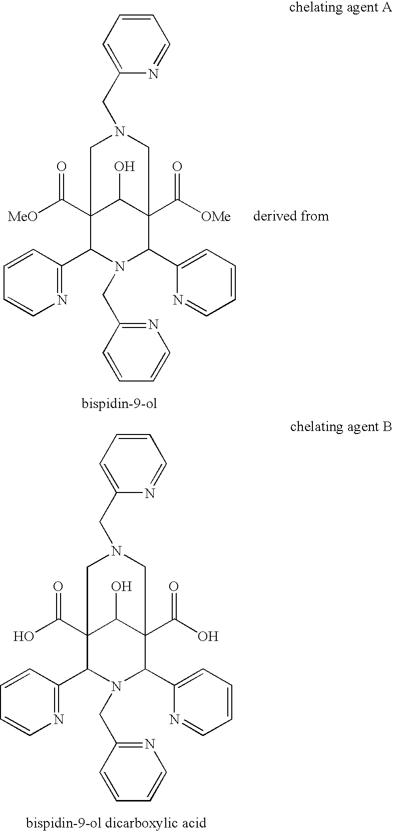

Chelating agent A can be referred to as (dimethyl 9-hydroxy-2-{(1Z)-1-[(1Z)-prop-2-en-1-ylideneamino]prop-1-en-1-yl}-4-pyridin-2-yl-3,7-bis(pyridin-2-ylmethyl)-3,7-diazabicyclo[3.3.1]nonane-1,5-dicarboxylate. Chelating agent B can be referred to as (9-hydroxy-2-{(1Z)-1-[(1Z)-prop-2-en-1-ylideneamino]prop-1-en-1-yl}-4-pyridin-2-yl-3,7-bis(pyridin-2-ylmethyl)-3,7-diazabicyclo[3.3.1]nonane-1,5-dicarboxylic acid.

Compounds of the general formula (M*×bisp) are obtained by adding an aqueous solution of the radioactive metal (M*) to an aqueous or aqueous/organic solution of a bispidine derivative (bisp), whereupon the metal complex is formed in more than 95% yield within a few minutes at room temperature or with gentle heating.

### EXAMPLE 1

### Chelating Agent A, Derived from Bispidin-9-Ol

500 mg of the appropriate bispidone derivative (0.844 mmol) are dissolved in 8.3 ml of dioxane. 5.8 ml of water are added to this solution. While cooling in ice and stirring, 0.216 g of NaBH₄ (5.71 mmol), dissolved in 4.5 ml of water, is added dropwise. The reaction solution is stirred while cooling in ice for a further 2 h and is stored in a refrigerator for 2 days. The pH of the solution is then adjusted to 1 with dilute HCl, and the reaction mixture is stored at room temperature for 2-3 h. The solution is then concentrated to one half and the pH is adjusted to 8 with 1M NaOH. It is subsequently extracted 5 times with CH₂Cl₂, and the organic phase is concentrated.

Yield: 60% of theory, m.p.: 167° C.

Elemental analysis (C₃₃H₃₃N₆O₅): calc. C, 66.77; H, 5.60; N, 13.48%. found C, 64.84; H, 6.03; N, 13.36%.

MS (ESI): 595 (M⁺)

### EXAMPLE 2

### Cu(II) Complex with Chelating Agent A

100 mg of chelating agent A (0.168 mmol) are heated in 2 ml of MeOH. A hot solution of 31.5 mg of Cu(NO₃)₂ (0.168 mmol) in 1 ml of MeOH is added thereto. The reaction mixture is cooled and the complex is precipitated with ether and separated off.

Yield: 21.9% (of theory)

Elemental analysis (C₃₃H₃₃N₈O₁₁Cu×H₂O): calc. C, 50.67; H, 4.38; N, 14.32%. found C, 49.53; H, 4.53; N, 14.00%.

Thin-layer chromatogram (RP-18, acetone/ammonium acetate (5%) 1:1): R_{f}=0.53

HPLC: (Jupiter Proteo 4μ 90 A; eluent A: CH₃CN+0.1% TFA, eluent B: H₂O+0.1% TFA,

10% A→70% A (t=20 min): t_{R}=10.76 min

### EXAMPLE 3

### Preparation of a ⁶⁴Cu-Labeled Complex with Chelating Agent A

10 μl of ⁶⁴CuCl₂ (0.1M HCl) are added to a solution which contains 0.1 mg of chelating agent A in 100 μl of 0.05M MES (morpholinoethanesulfonic acid)/NaOH buffer with a pH of 5.3. The solution is heated at 37° C. for 15 minutes.

Radiochemical purity: >95%

Thin-layer chromatogram (RP-18, acetone/ammonium acetate (5%) 1:1): R_{f}=0.53

HPL: (Jupiter Proteo 4μ 90 A; eluent A: CH₃CN+0.1% TFA, eluent B: H₂O+0.1% TFA,

10% A→70% A (t=20 min): t_{R}=10.76 min

### EXAMPLE 4

### Chelating Agent B

190 mg of potassium tert-butoxide (1.69 mmol) are introduced into a flask. 100 mg of chelating agent A (0.168 mmol) in 0.78 ml of dry THF are added thereto. Further addition of 1.5 ml of THF is followed by cooling the solution to 0° C. Then, while stirring vigorously, 15 μl of water are added dropwise. The solution is then kept under reflux for 8 h. Cooling is followed by neutralization with 1M HCl. The reaction solution is purified by passing through an RP-18 cartridge.

Yield: 25.5% of theory, m.p.: 174° C.

MS (ESI): 565 (M⁺)

### EXAMPLE 5

### Cu(II) Complex with Chelating Agent B

100 mg of chelating agent B (0.179 mmol) are heated in 2 ml of MeOH. A hot solution of 33.6 mg of Cu(NO₃)₂ (0.179 mmol) in 1 ml of MeOH is added thereto. The reaction mixture is cooled, and the complex is precipitated with ether and separated off.

Yield: 19.3% of theory.

Thin-layer chromatogram (RP-18, acetone/ammonium acetate (5%) 1:1): R_{f}=0.83

HPLC: (Jupiter Proteo 4μ 90 A; eluent A: CH₃CN+0.1% TFA, eluent B: H₂O+0.1% TFA,

10% A→70% A (t=20 min): t_{R}=9.73 min

### EXAMPLE 6

### Preparation of the ⁶⁴Cu Complex with Chelating Agent B

200 μl of 10⁻⁴M ligand solution (chelating agent B)—in MES (morpholinoethanesulfonic acid)/NaOH buffer with a pH of 5.4—were mixed with 50 μl of ⁶⁴CuCl₂ (0.1M HCl) (˜250 kBq) and shaken at room temperature for 1 min. Radiochemical purity: >99%

Thin-layer chromatogram (RP-18, acetonitrile+0.1% TFA/water+0.1% TFA 80:20) R_{f}=0.16

HPLC: (Jupiter Proteo 4μ 90 A; eluent A: CH₃CN+0.1% TFA, eluent B: H₂O+0.1% TFA,

10% A→70% A (t=20 min): t_{R}=9.40 min

### EXAMPLE 7

### Stability Investigations on the ⁶⁷Cu Complex with Chelating Agent B in Rat Plasma

500 μg of ligand B are dissolved in 50 μl of water/CH₃CN, and 50 μl of ⁶⁷CuCl₂ (0.1M HCl) (˜250 kBq) are added. 250 μl of phosphate buffer (Sörensen, pH=7.4) and 250 μl of rat plasma are added to this solution. It is then incubated at 37° C. for 2 h. Subsequently, cold EtOH (volume corresponds to the total volume of the solution) is added. Centrifugation at the highest setting for 5 minutes followed by removal of the EtOH phase and checking of the activity content (min. 100 kBq). A further 550 μl of cold EtOH is then added, and centrifugation is repeated. The EtOH solution is separated off from the precipitated proteins and concentrated. About 100 μl of water/CH₃CN solution (9:1) are added, and the mixture is acidified with 5 μl of TFA.

Thin-layer chromatogram (RT-18, acetonitrile+0.1% TFA/water+0.1% TFA 80:20)

HPLC: (Jupiter Proteo 4μ 90 A; eluent A: CH₃CN+0.1% TFA, eluent B: H₂O+0.1% TFA,

10% A→70% A (t=20 min)

1. A radioactive metal complex based on bispidine and the derivatives thereof as chelating agent having the following general formula (I) 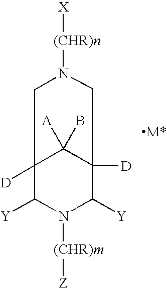
in which
A and B are independently of one another H, —OR¹, —SR¹, —NHR¹, —OC(O)R¹, —NHC(O)R¹ or a straight-chain or branched-chain (C₁₋₈) alkyl radical, or A and B together are ═O or ═S, where R¹ is hydrogen, a straight-chain or branched-chain (C₁₋₈) alkyl radical, a substituted or unsubstituted (C₆₋₁₀) aryl radical,
D is in each case a carboxyl group or a derivative derived therefrom and selected from esters, amides and peptides,
X, Y and Z are independently of one another an unsubstituted or substituted heterocycle selected from pyridine, pyrimidine, pyrazine, thiophene, furan, pyrazole, imidazole, thiazole, quinoline, quinazoline and quinoxaline, where the substituents may have been selected from straight-chain or branched-chain (C₁₋₈) alkyl radicals, halogen, straight-chain or branched-chain (C₁₋₈) alkoxy radicals, sulfonate, carboxylate, nitro, cyano, hydroxy, benzyl or phenyl,
the radical R in the respective alkylene bridge is independently of one another hydrogen, hydroxy, a straight-chain or branched-chain (C₁₋₈) alkyl radical, a straight-chain or branched-chain (C₁₋₈) alkoxy radical, —OC(O)R¹ or —NHC(O)R¹, where the radical R¹ is as defined above,
M* is a radioactive metal, and
m and n is in each case an integer between 1 and 5.

2. The radioactive metal complex as claimed in claim 1, wherein, in formula (I), A and B are independently of one another H, —OR¹, —OC(O)R¹ or —NHC(O)R¹, or A and B are together ═O,
D is in each case a carboxyl group or an ester derivative derived therefrom,
X, Y and Z are in each case an unsubstituted or substituted pyridine, and
the radical R in the respective alkylene bridge is independently of one another hydrogen, hydroxy, a straight-chain or branched-chain (C₁₋₈) alkyl radical, a straight-chain or branched-chain (C₁₋₈) alkoxy radical, —OC(O)R¹ or —NHC(O)R¹, where the radical R¹ is as defined above.

3. The radioactive metal complex as claimed in claim 1, wherein, in formula (I), A and B are respectively H and OH,
D is in each case a —COOH group or a —COOMe group,
X, Y and Z are in each case 2-pyridinyl, and
the radical R in the respective alkylene bridge is in each case hydrogen,
and m and n are each 1.

4. The radioactive metal complex as claimed in claim 1, wherein the radioactive metal M* is a nuclide of copper, of rare earths, Tc, In, Ga, Y or Re.

5. The radioactive metal complex as claimed in claim 1, wherein the radioactive metal M* is ⁶⁴Cu or ¹⁸⁸Re.

6. A method of nuclear medical diagnosis, comprising administering to a living body the radioactive metal complex as claimed in claim 1.

7. A method of internal radio nuclide therapy, comprising administering to a living body the radioactive metal complex as claimed in claim 1.

8. A method for preparing the radioactive metal complexes based on bispidine and the derivatives thereof as chelating agents as claimed in claim 1, in which a radioactive metal is mixed with the bispidine or derivative thereof in an aqueous or aqueous-organic solution and brought to a temperature between 20 and 50° C. and kept at this temperature for one hour.
